(19) United States
(12) Patent Application Publication
(10) Pub. No.: US 2005/0201989 A1
(43) Pub. Date: Sep. 15, 2005

(54) Compositions and methods for ex vivo preservation of blood vessels for vascular grafts using inhibitors of tumor necrosis factor-alpha

(75) Inventors: David M. Geliebter, Franklin Lakes, NJ (US); David H. Solomon, Brooklyn, NY (US)

Correspondence Address: JONES DAY, 222 EAST 41ST ST, NEW YORK, NY, 10017, US

(73) Assignee: St. Camillus Medical, Inc.

(21) Appl. No.: 11/060,615

(22) Filed: Feb. 17, 2005

Related U.S. Application Data
(60) Provisional application No. 60/546,454, filed on Feb. 19, 2004.

(51) Int. Cl. A61K 45/00; A01N 1/02

(52) U.S. Cl. 424/93.7; 514/323; 435/1.1

(57) ABSTRACT

The present invention relates to ex vivo methods for preserving/maintaining blood vessels that are to be used as vascular grafts by inhibiting TNF-α. The present invention also relates to compositions comprising an inhibitor of a TNF-α for use in the methods of the invention, which compositions are free of active TGF-β or does not contain active TGF-β in an amount greater than endogenous blood levels.

This application claims benefit under 35 U.S.C. § 119(e) of U.S. Provisional Application Ser. No. 60/546,454 filed Feb. 19, 2004, which is incorporated herein by reference in its entirety.

### 1. FIELD OF THE INVENTION

The present invention provides methods for the inhibition of tumor necrosis factor-alpha (TNF-α) in blood vessels, such as arteries or veins, as a means for preservation/maintenance of the blood vessel for use as a vascular graft prior to attachment to the recipient blood vessel in vascular surgical procedures. The present invention also provides compositions comprising an inhibitor of TNF-α for use in ex vivo preservation/maintenance of blood vessels useful as vascular grafts, which compositions are free of active transforming growth factor-beta (TGF-β) or does not contain active TGF-β in an amount greater than endogenous blood levels.

### 2. BACKGROUND OF THE INVENTION

2.1 Organ Preservation Solutions

Adequate preservation of organs intended for transplantation is critical to the proper functioning of the organ following implantation. Long preservation times are desired to enable cross-matching of donor and recipient to improve subsequent survival, as well as to allow for coast to coast and international transportation of organs to expand the donor and recipient pools. Organ preservation requires preservation of both the structure and function of the organ, including the specialized cells of the organ as well as the blood vessels and other cells found in the organ that together are responsible for its function.

Many different organ preservation solutions have been designed, as investigators have sought to lengthen the time that an organ may remain extra-corporeally, as well as to maximize function of the organ following implantation. Several of the key solutions that have been used over the years include: 1) the Stanford University solution [see, e.g., Swanson et al., 1988, Journal of Heart Transplantation, 7(6):456-467); 2) a modified Collins solution [see, e.g., Maurer et al., 1990, Transplantation Proceedings, 22(2):548-550; Swanson et al., supra]; and 3) the University of Wisconsin solution (U.S. Pat. No. 4,798,824, issued to Belzer et al.). Further, U.S. Pat. Nos. 5,552,267 and 5,370,989 to Stern et al. and Kayano et al., 1999, J. Thoracic Cardiovascular Surg. 118:135-144 describe an organ preservation solution known as the Columbia University solution.

In addition to the composition of the organ preservation or maintenance solution, the method of organ preservation also affects the success of preservation. Several methods of cardiac preservation have been studied in numerous publications: 1) warm arrest/cold ischemia; 2) cold arrest/macroperfusion; 3) cold arrest/microperfusion; and 4) cold arrest/cold ischemia. The first method involves arresting the heart with a warm cardioplegic solution prior to explantation and cold preservation, but this method fails because of the rapid depletion of myocardial energy store during the warm period. The second method, which involves arresting the heart with a cold preservation solution, is better; but continuous perfusion of the heart with preservation solution during the storage period fails because of the generation of toxic oxygen radicals. In addition, the procedure of the second method is cumbersome and does not lend itself to easy clinical use. The third method, called “trickle perfusion”, is better but also cumbersome. The fourth method of preservation is that of a cold cardioplegic arrest followed by a period of cold immersion of the heart. The fourth method is currently the standard method of cardiac preservation. This fourth method reliably preserves hearts for periods of up to six hours, but less than four hours is considered ideal for this method. Since a longer preservation time is desirable, attempts have been made to improve preservation solutions in such a way as to reliably preserve hearts and other organs for longer periods of time.

2.2 Coronary Artery Bypass Graft (CABG)

Coronary artery disease is a major medical problem throughout the world. Coronary arteries, as well as other blood vessels, frequently become clogged with plaque, impairing the efficiency of the heart's pumping action, and inhibiting blood flow which can lead to heart attack and death. In certain instances, these arteries can be unblocked through relatively noninvasive techniques such as balloon angioplasty. In other cases, a bypass of the blocked vessel is necessary.

A coronary artery bypass graft (“CABG”) involves performing an anastomosis on a diseased coronary artery to reestablish blood flow to an ischemic portion of the heart muscle. Improved long-term survival has been demonstrated bypassing the left anterior descending artery with a left internal mammary artery and this encouraged surgeons to extend revascularization with arterial grafts to all coronary arteries.

Since the internal mammary artery can only be used for two CABG procedures (using right and left internal mammary arteries, respectively), where multiple-vessels need to be bypassed, other arteries or veins have to be used. Such other arteries or veins that have been used include the right gastroepiploic artery, the inferior epigastric artery, the internal thoracic artery, the radial artery, and the saphenous vein. The internal mammary artery is the most common arterial conduit used for CABG; yet, despite its widespread use and superior patency when compared to the saphenous vein (Grondin et al., 1984, Circulation, 70 (suppl I): I-208-212; Camereon et al., 1996, N Engl J Med, 334:216-219), the saphenous vein continues to be one of the most popular conduits for CABG (Roubos et al., 1995, Circulation, 92 (9 Suppl) II31-6).

During a typical coronary artery bypass graft procedure using the saphenous vein, a section of the saphenous vein is surgically removed from the leg and the graft is retained ex vivo (out of the body) for a length of time prior to attachment to another blood vessel within the body (Angelini and Jeremy, 2002, Biorheology, 39(3-4):491-499). In a bypass operation involving such a venous graft, the graft is harvested by a surgically invasive procedure from the leg of the patient and then stored for up to about four hours ex vivo as the heart surgery is conducted. Although there are variations in methodology in surgical preparation of the heart, the first part of the procedure typically requires an incision through the patient's sternum (sternotomy), and in one technique, the patient is then placed on a bypass pump so that the heart can be operated on while not beating. In alternative techniques, the heart is not stopped during the procedure. Having harvested and stored the saphenous vein or arterial blood vessel conduit and upon completion of the surgery to prepare the heart for grafting, the bypass procedure is performed. A precise surgical procedure is required to attach the bypass graft to the coronary artery (anastomosis), with the graft being inserted between the aorta and the coronary artery. The inserted venous/arterial segments/transplants act as a bypass of the blocked portion of the coronary artery and thus provide for a free or unobstructed flow of blood to the heart. More than 500,000 bypass procedures are performed in the United States every year.

The overall short and long term success of the CABG procedure is dependent on several factors including the condition of the graft that is to be inserted which itself depends on any form of damage during the removal of the graft from the body or deterioration or damage of the graft due to storage conditions. In such circumstances, the short term detrimental effect can be potentially lethal thrombotic disease as a result of contact of flowing blood with a changed phenotype of the graft due to its deterioration or damage during the removal or storage stage. Possible long term detrimental effects include the vein graft itself becoming diseased, stenosed, or occluded, similar to the original bypassed vessel. In this case, the diseased or occluded saphenous vein grafts are associated with acute ischemic syndromes necessitating some form of intervention. It is, therefore, of critical importance not only that care be taken in the surgical procedure to remove the blood vessel to be used as the graft in surgical bypass procedures including CABG, but, also that no deterioration or damage occurs in the storage period of the graft prior to attachment to another blood vessel and the resumption of blood flow in that vessel.

2.3 Tumor Necrosis Factor-Alpha (TNF-α)

Tumor necrosis factor-alpha (TNF-α), also referred to as cachectin, was originally identified for its activity against tumor cells (Carswell et al., 1975, Proc. Natl. Acad. Sci. USA 72:3666-3670). Presently, TNF-α is recognized as a pleiotropic cytokine that functions in a complex and tightly regulated cytokine network. TNF-α activates multiple signal transduction pathways and induces or suppresses the expression of a wide variety of genes, including those that encode cytokines, adhesion molecules and inducible nitric oxide synthase. TNF-α has many pro-inflammatory actions, including orchestrating the inflammatory response through activation of expression of pro-inflammatory cytokine genes, such as IL-1 and IL-6, as well as its own production (Gordon et al., 1992, J. Immunol. 148:4021-4027). For a general review of TNF-α and its properties, see, e.g., Palladino et al., 2003, Nature Rev. Drug Discov. 2(9):736-746, and Azzawi and Haselton, 1999, Cardiovascular Research 43:850-859.

In order to decrease the likeliness of short and long term detrimental consequences of grafting blood vessels in surgical procedures, including coronary arterial bypass grafting (CABG), and consequently to improve the overall outcome of patients undergoing such procedures, there is a need for improved storing conditions for vascular grafts during the time period from harvesting of the graft to attaching the graft to another blood vessel in the patient.

Citation or identification of any reference in Section 2 or in any other section of this application shall not be construed as an admission that such reference is available as prior art to the present invention.

### 3. SUMMARY OF THE INVENTION

The present invention is directed to a solution comprising an inhibitor of TNF-α for preserving a blood vessel, or a functional portion thereof, removed from an individual, which solution is free of active transforming growth factor-beta (“TGF-β”) or does not contain active TGF-β in an amount greater than endogenous blood levels. The blood vessel or portion thereof can be used as a vascular graft in the same or different individual. It is believed that the use of a TNF-α inhibitor enhances the viability of the blood vessel and will result in less damage or phenotypic change of the blood vessel as a result of storage conditions. Thus, it is believed that blood vessels so treated will improve short and long term outcomes of vascular bypass procedures involving blood vessel grafts, including, but not limited to, coronary artery bypass, abdominal aneurysm repair, carotid endarterectomy, deep vein occlusion, or popliteal aneurysm repair.

Accordingly, the present invention provides a solution comprising an inhibitor of TNF-α and heparinized blood which solution is free of active transforming growth factor-beta (TGF-β) or does not contain active TGF-β in an amount greater than endogenous blood levels. Alternatively, the present invention provides a solution consisting of an inhibitor of TNF-α and heparinized blood, which solution is free of active TGF-β or does not contain active TGF-β in an amount greater than endogenous blood levels. The present invention also provides a method of preserving a blood vessel comprising contacting an isolated blood vessel or portion thereof ex vivo with a solution comprising an inhibitor of TNF-α, which solution is free of active TGF-β or does not contain active TGF-β in an amount greater than endogenous blood levels. The present invention also provides a method of preserving a blood vessel or a functional portion thereof comprising contacting an isolated blood vessel or portion thereof ex vivo with a solution consisting of an inhibitor of TNF-α, which solution is free of active TGF-β or does not contain active TGF-β in an amount greater than endogenous blood levels, said solution selected from the group consisting of heparinized blood, buffered saline, the Columbia University solution, the Euro-Collins solution, the University of Wisconsin solution, the low-potassium dextran glucose solution and the CELSIOR™ solution. In an embodiment where buffered saline, or the Columbia University solution, or the Euro-Collins solution, or the University of Wisconsin solution, or the low-potassium dextran glucose solution or the CELSIOR™ solution is used, preferably the solution is free of active TGF-β.

In other embodiments of the present invention, an isolated ex vivo blood vessel or functional portion thereof in contact with a solution comprising an inhibitor of TNF-α, which solution is free of active TGF-β or does not contain active TGF-β in an amount greater than endogenous blood levels is provided. As used herein, a functional portion of a blood vessel is a portion of a blood vessel of sufficient size as to be able to act as a vascular graft.

The present invention also provides a container containing a blood vessel or functional portion thereof in contact with a solution comprising an inhibitor of TNF-α, which solution is free of active TGF-β or does not contain active TGF-β in an amount greater than endogenous blood levels.

Further, the present invention provides a method of using a blood vessel as a vascular graft comprising contacting an isolated blood vessel or functional portion thereof ex vivo with a solution comprising an inhibitor of TNF-α, which solution is free of active TGF-β or does not contain active TGF-β in an amount greater than endogenous blood levels, and inserting the blood vessel into a patient so as to form a vascular graft in the patient. The present invention also provides a method for performing a coronary artery bypass graft in a patient comprising removing from contact with a blood vessel or functional portion thereof a solution comprising an inhibitor of TNF-α, which solution is free of active TGF-β or does not contain active TGF-β in an amount greater than endogenous blood levels; and grafting the blood vessel or functional portion thereof into the patient so as to serve as a coronary bypass graft.

As used herein transforming growth factor-beta (TGF-β) refers to any one of the three currently identified forms, TGF-β₁, TGFβ₂ and TGF-β₃, as well as other molecules related to this family of proteins, and those identified in the future, their alleles, and their predetermined amino acid sequence variants. See, for example, Derynck et al., 1985, Nature 316:701; European Patent Publication Nos. EP 200 341, EP 169 016, EP 268 561, and EP 267 463; U.S. Pat. No. 4,774,322; Seyedin et al., 1987, J. Biol. Chem. 262:1946-1949; Cheifetz et al., 1987, Cell 48:409-415; Jakowlew et al., 1988, Molecular Endocrin. 2:747-755; and Dijke et al., 1988, Proc. Natl. Acad. Sci. U.S.A. 85:4715-4719 for a discussion of the forms of TGF-β and of methods for its production and isolation as well as for determining its activity. TGF-β is secreted in virtually all cell types as a precursor in an inactive form and is proteolytically cleaved into an active form. Thus, as used herein, the active form of TGF-β is the mature form that has been proteolytically cleaved from its inactive precursor. For a general review of TGF-β and its activities, see, e.g., Sporn et al., 1987, J. Cell. Biol. 105:1039-1045 and Sporn and Roberts, 1988, Nature 322:217-219.

### 4. DETAILED DESCRIPTION OF THE INVENTION

It is believed that the use of a TNF-α inhibitor enhances the viability of a blood vessel and results in less damage or phenotypic change of the blood vessel as a result of storage conditions. Thus, blood vessels so treated should improve short and long term outcomes of vascular bypass procedures involving blood vessel grafts, including coronary artery bypass grafts, abdominal aneurysm repair, carotid endarterectomy, deep vein occlusion, and popliteal aneurysm repair. Exemplary blood vessels that can be so isolated include, but are not limited to, a saphenous vein, a mammary artery, a renal artery, and a radial artery.

4.1 Preservation Solutions

The present invention is directed to use of a solution comprising an inhibitor of TNF-α, which solution is free of transforming growth factor-beta (TGF-β) or does not contain TGF-β in an amount greater than endogenous blood levels. More preferably, the solution is free of active TGF-β or does not contain active TGF-β in an amount greater than endogenous blood levels. In specific embodiments, the solution can contain more than one inhibitor of TNF-α. The solution can be an aqueous solution or a semi-solid gel. In a preferred embodiment, the solution is an aqueous solution. Any physiologic solution to which the inhibitor is added can be used in the present invention so long as the solution does not damage the blood vessel graft tissue that is placed in it. For example, the solution comprising the inhibitor can be saline, buffered saline, phosphate buffered saline. The solution can also be Hank's Balanced Salt solution (HBSS), which typically comprises 1.26 mM CaCl₂, 5.36 mM KCl, 0.44 mM KH₂PO₄, 0.81 mM MgSO₄, 136 mM NaCl, 0.42 mM Na₂HPO₄, 6.1 mM glucose, 20 mM HEPES-NaOH, at pH 7.4 (Herreros et al., 2000, J Neurochem 74(5):1941-1950) or modified HBSS, which typically comprises 143 mM NaCl, 5.6 mM KCl, 2 mM MgCl₂, 10 mM HEPES, 10 mM glucose, 0.2 mM CaCl₂, and 0.4% BSA, at pH 7.2 (Briddon et al., 1999, Blood 93:3847-3855). The solution can also be Ringer's Lactate, which typically comprises 155 mM NaCl, 5 mM KCl, 2 mM CaCl₂, 1 mM MgCl₂, 2 mM NaH₂PO₄, 10 mM HEPES, and 10 mM glucose, at pH 7.2 (Sturgill-Koszycki and Swanson, 2000, J Exp. Med. 192:1261-1272). The solution can also be Tyrodes buffer, which typically comprises 137 mM NaCl, 12 mM NaHCO₃, 26 mM KCl, 5.5 mM glucose, 0.1% BSA, and 5.0 mM Hepes at pH 7.35 (Kasirer-Friede et al., 2002, J. Biol. Chem., 277:11949-11956). The solution can also be Kreb's buffer, which typically comprises 119 mM NaCl, 4.7 mM KCl, 2.5 mM CaCl₂, 1.17 MgSO₄, 25 mM NaHCO₃, 1.18 mM KH₂PO₄, 0.026 mM EDTA, and 5.5 mM glucose (Knock et al., 2002, J Physiology 538:879-890).

The solutions of the present invention can also contain a variety of additional additives, such as vasodilators, as detailed infra. In a preferred embodiment, the solution comprises the inhibitor of TNF-α activity; and heparinized blood, or buffered saline, or the Columbia University solution, or the Euro-Collins solution, or the University of Wisconsin solution, or the low-potassium dextran glucose solution, or the CELSIOR™ solution. Alternatively, the solution consists of an inhibitor of TNF-α; and heparinized blood, or buffered saline, or the Columbia University solution, or the Euro-Collins solution, or the University of Wisconsin solution, or the low-potassium dextran glucose solution, or the CELSIOR™ solution. In a preferred embodiment, the solution is sterilized.

In an embodiment of the present invention, the preservation solution further comprises a vasodilator in an amount sufficient to maintain vascular homeostasis. Preferably, the vasodilator is cell membrane permeable. The vasodilator can be selected from the group including, but not limited to, of adenosine 3′,5′-cyclic monophosphate (cyclic adenosine monophosphate, cyclic-AMP, or cAMP), analogues of adenosine 3′,5′-cyclic monophosphate (cyclic adenosine monophosphate, cyclic-AMP, or cAMP), guanosine 3′,5′-cyclic monophosphate (cyclic guanosine monophosphate, cyclic-GMP, or cGMP), analogues of guanosine 3′,5′-cyclic monophosphate (cyclic guanosine monophosphate, cyclic-GMP, or cGMP), nitroglycerin, adenosine, and pertussis toxin. Suitable combinations of the vasodilators may be used. The analogues of adenosine 3′,5′-cyclic monophosphate can be selected from the group including, but not limited to, dibutyryl adenosine 3′,5′-cyclic monophosphate (db cAMP), and 8-bromo-adenosine 3,″5′-cyclic monophosphate. Other suitable analogues of cAMP may also be used. Preferably, the analog is cell membrane-permeable. The optimal concentration of db cAMP is believed to be about 2 mM, though in specific embodiments, db cAMP concentrations of about 1 mM, or of about 2 to 4 mM can be used. It is known that db cAMP concentrations higher than about 4 mM become toxic to endothelial cells. Hence, 2 mM is considered to be the optimal concentration of db cAMP. In a preferred embodiment, the concentration of dibutyryl adenosine 3′,5′-cyclic monophosphate (db cAMP) ranges from about 1 mM to about 4 mM. The term “about” as used herein is intended to cover the range of experimental variation.

A preferred solution of the invention contains a vasodilator, wherein the vasodilator is dibutyryl adenosine 3′,5′-cyclic monophosphate (db cAMP), alone or in combination with nitroglycerin and adenosine. In another preferred solution, the vasodilator is 8-bromo-adenosine 3′,5′-cyclic monophosphate (8-bromo-cAMP), alone or in combination with nitroglycerin and adenosine. In a preferred embodiment, the concentration of nitroglycerin ranges from about 0.05 g/l to about 0.2 g/l. In another preferred embodiment, the concentration of adenosine ranges from about 3 mM to about 20 mM.

In certain embodiments, the solutions of the present invention also comprise a sugar, for example, D-glucose, e.g., in an amount sufficient to support intracellular function and maintenance of cellular bioenergetics. In a preferred embodiment, the concentration of the sugar ranges from about 50 mM to about 80 mM. The solutions can also comprise magnesium ions, e.g., in an amount sufficient to support intracellular function and maintenance of cellular bioenergetics. In a preferred embodiment, the concentration of magnesium ions ranges from about 2 mM to about 10 mM. In particular, the magnesium ions can be derived from magnesium sulfate, magnesium gluconate, or magnesium phosphate, or suitable combinations thereof. The magnesium ions can also be derived from some other suitable magnesium containing compound. D-Glucose, adenosine, and magnesium ions are substrates for adenosine triphosphate (ATP) synthesis. Metabolic substrates such as D-glucose and perhaps adenosine for ATP formation are probably important for maintaining the small degree of anaerobic metabolism that occurs during ex vivo preservation of blood vessels. Basal energy metabolism (even during hypothermia) can be supported by the anaerobic metabolism of D-glucose. The presence of magnesium ion allows for the proper functioning of the enzymes needed for adenosine triphosphate (ATP) synthesis. In general, substrates for ATP synthesis are helpful to allow intracellular function and maintenance of cellular bioenergetics.

In other embodiments, the preservation solution also comprises a macromolecule of molecular weight greater than 20,000 daltons, e.g., in an amount sufficient to maintain endothelial integrity and cellular viability. In a preferred embodiment, the macromolecule of molecular weight greater than 20,000 daltons is a macromolecule having a molecular weight greater than about 100,000 daltons, a polysaccharide, or a polyethylene glycol. Other suitable macromolecules can be used. The macromolecule of molecular weight greater than 20,000 daltons can be a colloid. In a preferred embodiment, the polysaccharide is a dextran. Furthermore, in a preferred combination, the dextran is a dextran molecule having a molecular weight of 308,000 daltons. Macromolecules of molecular weight greater than 20,000 daltons are believed to be helpful in reducing trans-endothelial leakage and subsequent intracellular and interstitial edema in the reperfusion period, by serving to plug small endothelial leaks which may occur. Macromolecules may thus also prevent the extravasation of intravascular contents into the pericellular space, thus helping to prevent cellular swelling and rupture during the preservation and recovery periods.

The osmolarity of the preservation solution of the invention is also a factor in helping to prevent cellular swelling and rupture. The osmolarity of the solution should be greater than the cellular osmolarity. Cellular osmolarity is about 290 mOSm/l. In a preferred embodiment, the osmolarity ranges from about 315 mOSm/l to about 340 mOSm/l.

The preservation solution also optionally comprises potassium ions, preferably in a concentration greater than about 110 mM. The potassium ions can be derived from potassium sulfate, potassium gluconate, monopotassium phosphate (KH₂PO₄), or suitable combinations thereof. The potassium ions can also be derived from some other suitable potassium containing compound. In a preferred embodiment, the concentration of potassium ions ranges from about 110 mM to about 140 mM.

In other embodiments, the preservation solution also comprises a buffer in an amount sufficient to maintain the average pH of the solution during the period of blood vessel preservation at about the physiologic pH value. In a preferred embodiment, the buffer is monopotassium phosphate (KH₂PO₄). However, other suitable buffers may be used. The buffering capacity should be adequate to buffer the organic acids that accrue during ischemia. Because basal metabolism results in the generation of acid, preferably a buffering system is used. The pH of the solution can decline during prolonged storage times that can be employed with this solution. In a preferred embodiment, the initial pH of the preservation solution is adjusted to the alkaline side of normal physiologic pH because then the average pH during storage of the blood vessel in the preservation solution remains physiologic. Normal physiologic pH is about 7.4. A preferred embodiment of the preservation solution has a pH range of about 7.3 to about 7.6. The pH may be adjusted to the desired value with the addition of a suitable base, such as potassium hydroxide (KOH). Hence, during the period of preservation, the pH of the preservation solution starts on the alkaline side of physiologic pH, and may drift slowly down to the acidic side of physiologic pH. But the average pH of the preservation solution during the period of preservation is preferably about the physiologic value.

In other embodiments, the preservation solution may further comprise impermeant anions, e.g., in an amount sufficient to help maintain endothelial integrity and cellular viability. The impermeant anion can be the gluconate anion or the lactobionate anion. Other suitable impermeant anions can be used. In a preferred embodiment, the concentration of the gluconate anion ranges from about 85 mM to about 105 mM. The gluconate anion can be derived from potassium gluconate or magnesium gluconate. The gluconate anion can also be derived from some other suitable gluconate containing compound. Impermeant anions are large anions that cannot cross cell membranes, so that sodium is at least in part prevented from diffusing down its concentration gradient into the cell during the preservation period. Impermeant anions thus help to prevent cellular edema.

The preservation solution may further comprise an anticoagulant, e.g., in an amount sufficient to help prevent clotting of blood within the capillary bed of the blood vessel. The anticoagulant can be heparin or hirudin. Other suitable anticoagulants may be used. In a preferred embodiment, the concentration of heparin ranges from about 1000 units/l to about 100,000 units/l. Anticoagulants are believed to help in preventing clotting of blood within the capillary bed of the preserved blood vessel. Specifically, anticoagulants are believed to help prevent a total no-reflow phenomenon at the level of the microcirculation, which would be undesirable following re-implantation and could result in graft failure. Anticoagulants are believed to be helpful in ensuring that thrombosis does not occur during or after preservation, so that nutrient delivery and toxin removal can proceed.

In yet another embodiment, the preservation solution may further comprise an antioxidant, e.g., in an amount sufficient to help decrease reperfusion injury secondary to oxygen free radicals. The antioxidant can be butylated hydroxyanisole (BHA), butylated hydroxytoluene (BHT), Vitamin C, Vitamin E, or suitable combinations thereof. Other suitable antioxidants can be used. In a preferred embodiment, the antioxidant is butylated hydroxyanisole (BHA) at a concentration range from about 25 μM to about 100 μM, alone or in combination with butylated hydroxytoluene (BHT) at a concentration range from about 25 μM to about 100 μM. The preservation solution can further comprise a reducing agent, e.g., in an amount sufficient to help decrease reperfusion injury secondary to oxygen free radicals. Any suitable reducing agent can be used.

Optionally, the preservation solution may further comprise N-acetylcysteine in an amount sufficient to help cells produce glutathione. In a preferred embodiment, the concentration of N-acetylcysteine ranges from about 0.1 mM to about 5 mM. N-acetylcysteine is an agent which can enter cells and is believed to play a role in helping cells to produce glutathione, which is a reducing agent. During blood vessel preservation, glutathione is lost. Simply adding glutathione to the preservation solution, however, would likely be of little to no help, because it is now known that glutathione in solution does not enter easily into the cell.

In another optional embodiment, the preservation solution may further comprise an agent that helps prevent calcium entry into cells in an amount sufficient to help prevent calcium entry into cells. Agents that help prevent calcium entry into cells include so-called calcium channel blockers, as well as other agents that serve the described function. An agent that helps prevent calcium entry into cells that can be used is verapamil. Other suitable agents that help prevent calcium entry into cells may be used. In a preferred embodiment, the concentration of verapamil ranges from about 2 μM to about 25 μM. Agents that help prevent calcium entry into cells are believed to play a role in preventing calcium overload.

Optionally, the solution does not contain sodium. The absence of sodium in the solution is preferred, since any sodium which may enter the cells during the period of preservation (when energy currency is low and the normal trans-cellular gradient may not be well maintained) may 1) lead to cellular swelling, 2) cause calcium entry by facilitated diffusion (following re-implantation), and 3) sodium load the cell, such that a high amount of energy is required following reestablishment of blood flow before a normal membrane potential can be re-established.

In other embodiments, the preservation solution can further comprise a bacteriostat, in an amount sufficient to help inhibit the growth of, or destroy, bacteria. The bacteriostat can be cefazolin or penicillin. Other suitable bacteriostats or antibiotics can be used. In a specific embodiment, the concentration of cefazolin ranges from about 0.25 g/l to about 1 g/l. The addition of an antibiotic to the solution is a surgical consideration, due in one embodiment to the practical inability of sterilizing the solution completely, as the high molecular weight solutes would not pass through a 0.2 micron membrane filter which may be used in the preparation of the preservation solution. It is believed that gamma irradiation may be used to better sterilize the solution.

In other embodiments, the solution can be any organ preservation solution known in the art in combination with the TNF-α inhibitor(s), which solution is free of active TGF-β or does not contain active TGF-β in an amount greater than endogenous blood levels. Illustrative examples of organ preservation solutions include, but are not limited to, the Euro-Collins solution, the University of Wisconsin solution, the low-potassium dextran glucose solution (PERFADEX™), the CELSIOR™ solution, and the Columbia University solution. In general, these solutions contain electrolytes and, optionally, sugars. One illustrative solution comprises a sugar in an amount sufficient to support intracellular function and maintenance of cellular bioenergetics, e.g., glucose, D-glucose; magnesium ions in an amount sufficient to support intracellular function and maintenance of cellular bioenergetics; potassium ions; and a buffer, e.g., a monopotassium phosphate or bicarbonate buffer, in an amount sufficient to maintain the average pH of the solution at about the physiologic pH value, i.e., about pH 7.3 to about pH 7.6. The specific composition of certain known solutions is listed below.

The Euro-Collins solution is described in Maurer et al., 1990, Transplantation Proceedings 22:548-550 and in Swanson et al., 1988, J Heart Transplantation 7(6):456-467, and typically comprises 115 mM potassium, 10 mM sodium, 8 mM magnesium, 10 mM bicarbonate, 100 mM phosphate, and 120 mM glucose, at a pH of about 7.4 and with an osmolality of about 452 mOsm/L.

The University of Wisconsin solution is described in U.S. Pat. No. 4,798,824 to Belzer et al., and typically comprises 125 mM potassium, 30 mM sodium, 5 mM magnesium, 25 mM phosphate, 5 mM sulfate, 100 mM lactobionate, 50 mM hydroxyethyl starch, 5 mM adenosine and 1 mM allopurinol, at a pH of about 7.4 and with an osmolality of about 327 mOsm/L.

The low-potassium dextran glucose solution (PERFADEX™), is commercially available from VitroLife, Gothenberg, Sweden, and typically comprises 4 mM potassium, 165 mM sodium, 2 mM magnesium, 101 mM chloride, 34 mM phosphate, 2 mM sulfate, 20 mM Dextran-40, and 56 mM glucose, at a pH of about 7.4 and with an osmolality of about 335 mOsm/L.

The CELSIOR™ solution is commercially available from Sangstat Medical Corporation, Freemont, Calif., and typically comprises 60 mM mannitol, 80 mM lactobionic acid, 20 mM glutamic acid, 30 mM histidine, 0.25 mM calcium, 15 mM potassium, 13 mM magnesium, 100 mM sodium hydroxide, and 3 mM reduced glutathione, at a pH of about 7.3 and with an osmolality of about 320-360 mOsm/L.

The Columbia University solution is described in U.S. Pat. Nos. 5,370,989 and 5,552,267 to Stern et al., and typically comprises 120 mM potassium, 5 mM magnesium, 25 mM phosphate, 5 mM sulfate, 95 mM gluconate, 50 mM Dextran 50, 67 mM glucose, 5 mM adenosine, 2 mM dibutyryl adenosine 3′,5′-cyclic monophosphate (db cAMP), 0.1 mg/ml nitroglycerin, 50 μM butylated hydroxyanisole, 50 mM butylated hydroxytoluene, 0.5 mM N-acetylcysteine, 10 U/ml heparin, and 10 μM verapamil, at a pH of about 7.6 and with an osmolality of about 325 mOsm/L.

In a specific embodiment, a preservation solution of the present invention comprises, or alternatively consists of, an inhibitor of TNF-α; a vasodilator in an amount sufficient to maintain vascular homeostasis, wherein the vasodilator is selected from the group consisting of: adenosine 3′,5′-cyclic monophosphate analogues, guanosine 3′,5′-cyclic monophosphate analogues, nitroglycerin, and pertussis toxin; a sugar in an amount sufficient to support intracellular function and maintenance of cellular bioenergetics; magnesium ions in an amount sufficient to support intracellular function and maintenance of cellular bioenergetics; a macromolecule of molecular weight greater than 20,000 daltons in an amount sufficient to maintain endothelial integrity and cellular viability; potassium ions in a concentration greater than about 110 mM; and a buffer in an amount sufficient to maintain the average pH of the blood vessel or portion thereof during said contacting step at about the physiologic pH value, which solution is free of active TGF-β or does not contain active TGF-β in an amount greater than endogenous blood levels.

In another specific embodiment, a preservation solution of the present invention comprises, or alternatively consists of, an inhibitor of TNF-α; 67.4 mM D-glucose; 5 mM magnesium sulfate (MgSO₄); 25 mM monopotassium phosphate (KH₂PO₄); 50 g/l dextran (molecular weight 308,000 daltons); 95 mM potassium gluconate (K-gluconate); 50 μM butylated hydroxyanisole (BHA); 50 μM butylated hydroxytoluene (BHT); 0.5 mM N-acetylcysteine; 5 mM adenosine; 0.1 g/l nitroglycerin; 10 μM verapamil; 2 mM dibutyryl adenosine 3′,5′-cyclic monophosphate (db cAMP); 10,000 units heparin; and 0.5 g/l cefazolin, which solution is free of active TGF-β or does not contain active TGF-β in an amount greater than endogenous blood levels. The pH is adjusted to 7.6 with potassium hydroxide.

The amount of the preservation solution required in a surgical procedure, such as a cardiac arterial bypass graft (CABG) would be clear to one who is skilled in such surgical procedures, and depends, inter alia, upon the particular container used to hold the blood vessel and solution.

The preservation solution is suitable for use at the low temperatures that may be required or desired during vascular bypass, e.g., CABG, or other surgical procedure. For instance, temperatures of about 0.5 to about 10 degrees Centigrade, preferably 4° C., may be used during CABG or other surgical procedure.

The present invention is also directed to a container containing a preservation solution of the present invention. In a preferred embodiment, the container is one of certain dimensions useful in preserving a blood vessel. In another embodiment, the container also contains the blood vessel or functional portion thereof, intended for use as a vascular graft, in contact with the solution.

4.2 Tumor Necrosis Factor-Alpha (TNF-α) Inhibitors

The preservation solutions of the present invention comprise one or more inhibitor(s) of TNF-α. In a specific embodiment, the inhibitor of TNF-α binds to TNF-α. In another embodiment, the inhibitor of TNF-α binds to the TNF-α receptor, thereby blocking binding of TNF-α to its receptor. In yet another embodiment, the inhibitor of TNF-α inhibits the production or expression of TNF-α, or otherwise reduces the levels of TNF-α. Any method known in the art can be used to measure the activity of TNF-α. Illustrative methods for measuring TNF-α activity are described in Chimalakonda and Mehvar, 2003, Pharmaceutical Research 20(7):1001-1008, Meldrum et al., 1997, Immunology 92:472-477 (measuring TNF-α by immunoassay, e.g., ELISA); Khimenko et al., 1998, J. App. Physiol. 85(6):2005-2011, Wallach, 1984, J. Immunol. 132:3884-3893, Yuhas et al., 1990, Eur. Cytokine Netw. 1:35-40 (measuring TNF-α levels by a cell cytotoxicity assay); Ueland et al., 2003, Am. J. Cardiol. 92:715-717 (quantifying TNF-α messenger RNA by PCR).

In a specific embodiment, the inhibitor is present in the solution in a concentration range of about 0.05 μM to about 250 μM, preferably in a range of about 0.1 μM to about 100 μM. In another embodiment, when the inhibitor is an anti-TNF-α antibody, the antibody is present in the solution in a concentration range of about 0.1 μg/ml to about 100 μg/ml. In an embodiment, the concentration range refers to the concentration of a single inhibitor in the solution. The optimal concentration of the inhibitor in the solution to achieve inhibition can be determined by standard techniques.

Inhibitors of TNF-α activity are known in the art, such as antibodies to TNF-α (e.g., neutralizing antibodies), soluble TNF-α receptors, thalidomide, and other small molecules. For example, there are FDA-approved anti-TNF-α antibodies that are commercially available, such as etanercept (ENBREL™) available from Amgen, Inc. Thousand Oaks, Calif.; infliximab (REMICADE™) available from Centocor, Inc. Malvern, Pa.; and adalimumab (HUMIRA™) available from Abbott Laboratories, Abbott Park, Ill. Etanercept is a soluble TNF-α receptor fusion protein (type II TNF-alpha receptor conjugated to the Fc portion of IgG1) with the ability to bind and neutralize the activity of soluble TNF-α. Infliximab is a chimeric (human/mouse) anti-TNF-α monoclonal IgG1 kappa antibody, which neutralizes soluble TNF-α and blocks membrane bound cytokine. Adalimumab is a fully human anti-TNF-α monoclonal antibody.

Other exemplary TNF-α inhibitors include, but are not limited to, soluble TNF-receptors, such as the polyethylene glycol-derivatized soluble tumor necrosis factor-receptor type I under going clinical trials by Amgen, Inc. (PEG-sTNF-RI); and a dominant negative TNF (DN-TNF) which is a variant TNF ligand that has been engineered to eliminate affinity for the receptor yet retains affinity for other ligand monomers such that upon administration exchange of DN-TNF with native endogenous homotrimers depletes the pool of active TNF ligand, replacing it with heterotrimers that do not agonize the TNF receptor. Yet other exemplary inhibitors include, but are not limited to, thalidomide, which presumably reduces the level of TNF-α; selective cykotine inhibitory drugs (SelCIDs) such as those determined to have an inhibitory effect on phosphodiesterase type 4 enzyme (PDE-4), which results in decreased TNF-α production; and other immunomodulatory drugs (IMiDs) which inhibit TNF-α production.

Other illustrative inhibitors of TNF-α are described in Palladino et al., 2003, Nature Rev. Drug Discov. 2(9):736-746; U.S. Pat. No. 5,817,670 to Takayama et al.; European Patent Publication No. 0 387 095 A1; Arias-Diaz et al., 1994, Transplantation Proceedings 26(2):698-700; Hirokawa et al., 2002, Transplantation 74:779-783; Meldrum et al., 1997, Immunology 92:472-477; Chimalakonda and Mehvar, 2003, Pharmaceutical Research 20(7):1001-1008; Currin et al., 1993, Transplantation Proceedings 25(1):1631-1632; European Patent Publication No. 1 208 748 A1; U.S. Pat. No. 6,040,328 to Tailhan-Lomont et al.; U.S. Pat. No. 5,672,347 to Aggarwal et al.; International Patent Publication No. WO 2001/58473 A1; U.S. Patent Publication No. 2003/0096737 A1 to Diu-Hercend et al.; and U.S. Pat. No. 6,423,710 B1 to Martins et al. The disclosures of each of the foregoing citations are incorporated by reference herein in their entirety. In a preferred embodiment, illustrative examples of inhibitors of TNF-α activity include, but are not limited to, etanercept (ENBREL™), infliximab (REMICADE™), adalimumab (HUMIRA™), thalidomide, PEG-sTNF-RI, DN-TNF, and a mixture of any two or more of the foregoing.

4.3 Methods of Preservation and Use of Preserved Blood Vessels

The invention also provides a method of preserving or maintaining a blood vessel comprising contacting the blood vessel with a solution of the present invention comprising an inhibitor of TNF-α, which solution is free of active TGF-β or does not contain active TGF-β in an amount greater than endogenous blood levels. The contacting comprises immersing, infusing, flushing, or perfusing. Other suitable procedures of contacting can be used. The method can be used wherein the blood vessel is intended for transplantation for a vascular bypass procedure, e.g., abdominal aneurysm repair, carotid endarterectomy, deep vein occlusion, popliteal aneurysm repair, or for a coronary arterial bypass (CABG). Hence, the preservation solution may be used to preserve a blood vessel or functional portion thereof prior to use in such vascular transplantation procedures.

Any known blood vessel or a functional portion thereof can be preserved ex vivo in solution of the invention, preferably prior to use as a vascular graft. The blood vessel can be an artery or a vein. Exemplary blood vessels include, but are not limited to, the internal mammary artery, the renal artery, the radial artery, the internal thoracic artery, the right gastroepiploic artery, the inferior epigastric artery and the saphenous vein, or a functional portion thereof. Preferably, the blood vessel is a saphenous vein or functional portion thereof. For example, where the blood vessel graft is for a coronary arterial bypass, the blood vessel can be the internal mammary artery, the radial artery, the internal thoracic artery, the right gastroepiploic artery, the inferior epigastric artery and the saphenous vein, or a functional portion of the artery or vein. Preferably, the blood vessel for use as a graft is the saphenous vein or a functional portion thereof. In another example, where the graft is for abdominal aneurysm repair, carotid endarterectomy, deep vein occlusion, or popliteal aneurysm repair, the blood vessel is the renal artery or functional portion thereof, or the saphenous vein or a functional portion thereof. Preferably, the graft is isolated from the saphenous vein or a functional portion thereof.

As used herein, a “functional” portion of a blood vessel refers to a portion that is able to act as a vascular graft. The blood vessel can be isolated from and used as a vascular graft in, e.g., any mammal including primates, pigs, dogs, cats. Preferably, the blood vessel is isolated from a human, e.g., human child (less than 18 years old), or human adult (18 years or older). Preferably, the blood vessel is isolated from the patient in which it is subsequently used as a vascular graft.

One embodiment of the invention is directed to a method of preserving a blood vessel comprising contacting an isolated blood vessel or portion thereof ex vivo with a solution comprising an inhibitor of TNF-α, which solution is free of active TGF-β or does not contain active TGF-β in an amount greater than endogenous blood levels. Another embodiment is directed to a method of preserving a blood vessel comprising contacting an isolated blood vessel or portion thereof ex vivo with a solution consisting of an inhibitor of TNF-α, which solution is free of active TGF-β or does not contain active TGF-β in an amount greater than endogenous blood levels, said solution selected from the group consisting of heparinized blood, buffered saline, the Columbia University solution, the Euro-Collins solution, the University of Wisconsin solution, the low-potassium dextran glucose solution and the CELSIOR™ solution. In a preferred embodiment, the method of preserving a blood vessel comprising contacting an isolated blood vessel or portion thereof ex vivo with a solution comprising an inhibitor of TNF-α, which solution is free of active TGF-β or does not contain active TGF-β in an amount greater than endogenous blood levels and is at a temperature in the range of about 0.5° C. to about 10° C., preferably at 4° C., during the contacting step. In another preferred embodiment, the contacting is for a time period of not longer than 4 hours, preferably not longer than 2 or 3 hours.

Optionally, prior to use of the blood vessel or functional portion thereof as a vascular graft, the method further comprises a step of removing the solution from contact with the blood vessel or portion thereof. Preferably, the removing of the solution comprises flushing, immersing, infusing, or perfusing the blood vessel or portion thereof with a second solution that lacks the TNF-α inhibitor. Preferably, the second solution is appropriate for maintaining cardiovascular homeostasis in vivo, e.g., the solution lacks potassium. An exemplary solution is saline or Ringer's Lactate.

In another embodiment, the present invention is directed to an isolated ex vivo blood vessel or functional portion thereof in contact with a solution comprising an inhibitor of TNF-α, which solution is free of active TGF-β or does not contain active TGF-β in an amount greater than endogenous blood levels, preferably at a temperature in the range of about 0.5° C. to about 10° C., more preferably 4° C. The present invention is also directed to an isolated ex vivo isolated blood vessel or functional portion thereof in contact with a solution comprising, or alternatively consisting of, an inhibitor of TNF-α, and is free of active TGF-β or does not contain active TGF-β in an amount greater than endogenous blood levels. Preferably, the contacting is at a temperature in the range of about 0.5° C. to about 10° C., and more preferably at 4° C. Any known blood vessel or a functional portion thereof can be preserved ex vivo in solution of the invention, preferably prior to use as a vascular graft. Exemplary blood vessels include, but are not limited to, the internal mammary artery, the radial artery, the internal thoracic artery, the right gastroepiploic artery, the inferior epigastric artery and the saphenous vein. As used herein, a “functional” portion of a blood vessel refers to a portion that is able to act as a vascular graft. The blood vessel can be isolated from, e.g., any mammal including primates, pigs, dogs, cats. Preferably, the blood vessel is isolated from a human, e.g., human child (less than 18 years old), or human adult (18 years or older).

In another embodiment, the invention is directed to a container containing a solution of the invention comprising the inhibitor of TNF-α and the blood vessel or functional portion thereof, which solution is free of active TGF-β or does not contain active TGF-β in an amount greater than endogenous blood levels. Preferably, the blood vessel or portion thereof is a human blood vessel or portion thereof.

In yet another embodiment, the present invention is directed to a method of using a blood vessel as a vascular graft comprising contacting an isolated blood vessel or functional portion thereof ex vivo with a solution comprising an inhibitor of TNF-α, which solution is free of active TGF-β or does not contain active TGF-β in an amount greater than endogenous blood levels, and inserting the blood vessel into a patient so as to form a vascular graft in the patient. In one embodiment, the solution further comprises heparinized blood. Preferably, the temperature of the solution ranges from about 0.5° C. to about 110° C., and more preferably is about 4° C. Preferably, the contacting is for a time period not longer than four hours. The method can further comprise a step before inserting the vessel of removing the solution from contact with the blood vessel or portion thereof, wherein the removing step comprises flushing, immersing, infusing, or perfusing the blood vessel or portion thereof with a second solution that lacks the TNF-α inhibitor. Preferably, the second solution is appropriate for maintaining cardiovascular homeostasis in vivo, e.g., the solution lacks potassium. An exemplary solution is saline or Ringer's Lactate.

Any known blood vessel or a functional portion thereof can be preserved ex vivo in a solution of the invention, preferably prior to use as a vascular graft. Exemplary blood vessels include, but are not limited to, the internal mammary artery, the renal artery, the radial artery, the internal thoracic artery, the right gastroepiploic artery, the inferior epigastric artery and the saphenous vein. Preferably, the blood vessel or functional portion thereof is a saphenous vein or functional portion thereof. As used herein, a “functional” portion of a blood vessel refers to a portion that is able to act as a vascular graft. The blood vessel can be isolated from, e.g., any mammal including primates, pigs, dogs, cats. Preferably, the blood vessel is isolated from a human, e.g., human child (less than 18 years old), or human adult (18 years or older). Further, the blood vessel or portion thereof is isolated from the same patient receiving the graft, i.e., the graft is autologous.

In a particular embodiment, the inhibitor is etanercept (ENBREL™), infliximab (REMICADE™), adalimumab (HUMIRA™), thalidomide, PEG-sTNF-RI, DN-TNF, or a mixture of any two or more of the foregoing.

In specific embodiments, the solution is the Columbia University solution further comprising said inhibitor, or the Euro-Collins solution further comprising said inhibitor, or the University of Wisconsin solution further comprising said inhibitor, or the low-potassium dextran glucose solution further comprising said inhibitor, or the CELSIOR™ solution further comprising said inhibitor. Optionally, the solution further comprises a vasodilator. Exemplary vasodilators include, but are not limited to an analog of adenosine 3′,5′-cyclic monophosphate or an analog of guanosine 3′,5′-cyclic monophosphate, such as dibutyryl adenosine 3′,5′-cyclic monophosphate (db cAMP) or 8-bromo-adenosine 3′,5′-cyclic monophosphate (8-bromo-cAMP). Preferably, the analog is cell membrane-permeable. Other vasodilators include, but are not limited to nitroglycerin, adenosine, pertussis toxin.

In yet another embodiment, the present invention is directed to a method for performing a coronary artery bypass graft in a patient comprising, removing from contact with a blood vessel or functional portion thereof a solution comprising an inhibitor of TNF-α, which solution is free of active TGF-β or does not contain active TGF-β in an amount greater than endogenous blood levels; and grafting the blood vessel or functional portion thereof into the patient so as to serve as a coronary bypass graft. Preferably, the patient is a human patient and the blood vessel or portion thereof was isolated from the same patient. Alternatively, the blood vessel is isolated from a non-human animal.

Another embodiment of the invention is directed to a pharmaceutical pack or kit comprising one or more containers filled with a solution of the invention comprising an inhibitor of TNF-α, which solution is free of active TGF-β or does not contain active TGF-β in an amount greater than endogenous blood levels. For example, the kit can comprise a container containing the low-potassium dextran glucose solution (PERFADEX™) further comprising an inhibitor of TNF-α. Optionally associated with such container(s) can be instructions for use of the kit and/or a notice in the form prescribed by a governmental agency regulating the manufacture, use or sale of pharmaceuticals or biological products, which notice reflects approval by the agency of manufacture, use or sale for human administration.

The following series of examples are presented by way of illustration and not by way of limitation on the scope of the present invention.

### 5. EXAMPLE

5.1 In carrying out a coronary bypass using a saphenous vein as the vascular graft, the patient is first anesthetized and a portion of the saphena is excised from either leg. The excised saphenous vein is placed in contact with a preservation solution comprising an inhibitor of TNF-α in a kidney dish, which solution is free of active TGF-β or does not contain active TGF-β in an amount greater than endogenous blood levels, such that the solution is both inside and outside the vein and the dish is placed on ice. The excision in the leg is closed, and, concurrently, the chest is opened to allow access to the heart. The patient is placed on life support with a cardiac bypass machine and the heart is stopped. The saphenous vein is removed from the solution and is rinsed (flushed) with buffered saline lacking the TNF-α inhibitor and potassium ions. The saphenous vein is cut to size for the bypass area and is grafted onto the cardiac tissue. The inserted venous segment acts as a bypass of the blocked portion of the coronary artery, and, thus, provides for a free or unobstructed flow of blood to the heart. The patient's heart is restarted and the chest is closed.

5.2 The following experimental vein graft procedure is used to assess the efficacy of a composition of the invention in ex vivo preservation of a blood vessel to be used as a vascular graft.

Vein Graft Procedure—Intrapositional Saphenous Vein Anastamosis

Female sheep, dogs or pigs are purchased from Charles River (Charles River, Mass.). The investigation will conform with the Guide for the Care and Use of Laboratory Animals published by the US National Institutes of Health (NIH Publication No. 85-23, revised 1996). The surgical procedures used are standard; animals are anesthetized by intraperitoneal injection of ketamine (50 mg/kg) and xylazine (5 mg/kg). An incision is made on the left and right legs to expose the saphenous vein and femoral artery. A segment of the saphenous vein (19 cm long) is transected after ligation at both ends with 8-0 sutures. This segment is washed with saline solution containing 100 U/ml of heparin, and stored at 25° C. for up to 2 hours in heparinized saline or in an experimental solution of heparinized saline containing a TNF-α inhibitor of the invention, such as etanercept (ENBREL™).

A portion of the graft before these incubation is cut (0.5 cm) and placed in formaldehyde fixative (10%); after incubation for up to 2 hours, 0.5 cm sections are also cut and placed in formaldehyde fixative (10%), both for later immunohistochemical analysis as described, infra. After incubation in control or experimental solutions as described, a segment of the femoral artery is temporally occluded at two places with a microvascular clamp (Roboz Surgical Instrument Co., Gaithersburg, Md.), and a circular incision (of about the same size as the vein in diameter) is made. The anastamosis in loop is repaired by suturing the prepared vein into the clamped femoral artery with an 11-0 continuous suture around vein graft artery anastamosis. Contact between the instruments and the vein graft endothelium is avoided as much as possible throughout the procedure. After the vascular clamp is removed, the vein is inspected for adequacy of repair. Surgery is considered successful if strong pulsation is confirmed in both the graft and native artery without significant bleeding. If there is no pulsation or pulsations are diminished within a few minutes of restoration of blood flow, the procedure is considered a surgical failure. Cefazolin (50 mg/kg,) is administered and the skin incision is closed with a 6-0 nylon suture. Buprenorphine (2.5 mg/kg) is given subcutaneously for postoperative analgesia. The duration of the entire procedure is approximately 30 minutes. One leg in each animal is for the experimental solutions; the contralateral leg is always used for the control solution. In order to verify intimal hyperplasia, both phorbol myristate acetate (PMA, Sigma, St. Louis, Mo.) and lipopolysaccaride (LPS, Sigma, St. Louis, Mo.) at 1.0 μM is used to incubate saphenous vein segments for up to 2 hours as positive controls.

Morphology

Animals are sacrificed at various time points after surgery and perfusion-fixed using 10% formaldehyde at physiological pressure. The grafts, together with a short segment of the native femoral artery, are harvested and cut at the center. The specimens are embedded in medium (OCT compound), and frozen at −80° C. The section (5 μm) at the mid portion of each composite graft is stained with hematoxylin and eosin (H&E) or Van Gieson's elastic stain (Sigma, St. Louis, Mo.), and the degree of neointimal expansion is analyzed quantitatively using a Zeiss microscope and image analysis system (Media Cybernetics. Silver Spring, Md.). The consistency of neointimal formation in the central portion of the graft is histologically confirmed by analyzing serial sections from the center to the proximal and distal ends of the graft. The neointima of the vein graft is defined as the region between the lumen and the adventitia. Neointimal cell number is calculated by counting the number of nuclei visible in sections stained with H&E. The percentage of neointimal expansion is calculated as 100×(neointimal area/neointimal area+luminal area). These quantifications are performed by an observer blinded to the experimental circumstances. Masson Trichrome stain is performed according to the manufacture's instructions (Sigma, St. Louis, Mo.).

En Face Immunofluorescence

The procedure used in this study is similar to that reported by Zou et al., 2000, Circ Res 86:434-440 and Dietrich et al., 2000, Arterioscler Thromb Vasc Biol 20:343-352. The vein patch is retrieved 24 hours after surgery and mounted onto a glass slide with endothelium side up, and air-dried for 1 hour at room temperature. The segments are fixed in cold acetone (−20° C.) for 10 minutes and rinsed in PBS. The segments are then incubated with rat monoclonal antibody to MAC-1 (1:25, Pharmingen, San Diego, Calif.) for 30 minutes and visualized with FITC-labeled rabbit anti-rat IgG (1:25, Sigma, St. Louis, Mo.). MAC-1 positive sells are blindly counted at ×400 magnification in 10 fields of each segment.

Immunohistochemistry

Representative sections (5 mm) are immunostained with rat anti PECAM-1 (CD31) antibody (1:100, Pharmingen, San Diego, Calif.), rat anti-MAC-1 antibody (1:50, Pharmingen, San Diego, Calif.), and hamster anti-ICAM-1 antibody (1:100, Pharmingen, San Diego, Calif.). Sections are blocked with hydrogen peroxide (0.3%) in methanol for 10 min. Blocking is performed with goat serum (4%) and bovine serum albumin (1%) in PBS. Primary antibodies are added to slides, and incubated overnight at 4° C. Secondary antibodies (1:100; anti-hamster or rat IgG, Phamingen, San Diego, Calif.) are added for 30 min at room temperature. Sections are reacted with horseradish peroxidase conjugated streptavidin (1:100, Sigma, St. Louis, Mo.) for 30 min at room temperature and developed with 3.3′-diaminobenzidine (DAB substrate kit, Vector, Burlingame, Calif.).

Statistical Analysis

All data are expressed as mean +/− SEM. Student's unpaired t test for a comparison between two groups, or ANOVA with post hoc analysis using the Bonferroni/Dunn test for a comparison among more than two groups are used to determine significant difference. P values of less than 0.05 are considered statistically significant. All analyses are performed using the Statview statistical package, version J5.0 (Abacus Concepts Inc., Berkeley, Calif.).

### 6. REFERENCES CITED

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

1. A method of using a blood vessel as a vascular graft comprising:
(a) contacting an isolated blood vessel or functional portion thereof ex vivo with a solution comprising an inhibitor of TNF-α, which solution is free of active TGF-β or does not contain active TGF-β in an amount greater than endogenous blood levels, and;
(b) inserting the blood vessel into a patient so as to form a vascular graft in the patient.

2. The method of claim 1, wherein the temperature of the solution ranges from about 0.5° C. to about 10° C.

3. The method of claim 1, wherein said contacting step is for a time period not longer than four hours.

4. The method of claim 1, which further comprises before step (b) a step of removing the solution from contact with the blood vessel or portion thereof.

5. The method of claim 4, wherein said removing step comprises flushing the blood vessel or portion thereof with a second solution lacking said inhibitor.

6. The method of claim 5, wherein said second solution is buffered saline or Ringer's Lactate.

7. The method of claim 1, wherein the blood vessel is a saphenous vein, a mammary artery, or a radial artery; and the vascular graft is a coronary artery bypass graft.

8. The method of claim 7, wherein the blood vessel is a saphenous vein.

9. The method of claim 1, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

10. The method of claim 1, wherein said solution further comprises heparinized blood.

11. The method of claim 1, wherein said solution further comprises buffered saline.

12. The method of claim 1, wherein said solution is the Columbia University solution further comprising said inhibitor.

13. The method of claim 1, wherein said solution is the Euro-Collins solution further comprising said inhibitor.

14. The method of claim 1, wherein said solution is the University of Wisconsin solution further comprising said inhibitor.

15. The method of claim 1, wherein said solution is the low-potassium dextran glucose solution further comprising said inhibitor.

16. The method of claim 1, where said solution is the CELSIOR™ solution further comprising said inhibitor.

17. The method of claim 1, wherein said solution further comprises an analog of adenosine 3′,5′-cyclic monophosphate or an analog of guanosine 3′,5′-cyclic monophosphate.

18. The method of claim 1, wherein said solution further comprises dibutyryl adenosine 3′,5′-cyclic monophosphate (db cAMP).

19. The method of claim 1, wherein said solution further comprises 8-bromo-adenosine 3′,5′-cyclic monophosphate (8-bromo-cAMP).

20. The method of claim 1, wherein said solution further comprises nitroglycerin.

21. The method of claim 1 wherein said solution further comprises:
(a) a vasodilator in an amount sufficient to maintain vascular homeostasis, wherein the vasodilator is selected from the group consisting of: adenosine 3′,5′-cyclic monophosphate analogues, guanosine 3′,5′-cyclic monophosphate analogues, nitroglycerin, and pertussis toxin;
(b) a sugar in an amount sufficient to support intracellular function and maintenance of cellular bioenergetics;
(c) magnesium ions in an amount sufficient to support intracellular function and maintenance of cellular bioenergetics;
(d) a macromolecule of molecular weight greater than 20,000 daltons in an amount sufficient to maintain endothelial integrity and cellular viability;
(e) potassium ions in a concentration greater than about 110 mM; and
(f) a buffer in an amount sufficient to maintain the average pH of the blood vessel or portion thereof during said contacting step at about the physiologic pH value.

22. The method of claim 9, wherein the concentration of the inhibitor TNF-α ranges from about 0.1 μM to 100 μM.

23. The method of claim 1, wherein said solution further comprises Phosphate Buffered Saline (PBS), Hanks' Balanced Salt Solution (HBSS), HBSS (Modified), Ringer's Lactate, Tyrodes buffer, Krebs buffer, Euro-Collins solution, University of Wisconsin solution, low-potassium dextran glucose solution, CELSIOR™ solution, or Columbia University solution.

24. The method of claim 1, wherein the patient is a human.

25. The method of claim 24, wherein the blood vessel or portion thereof is from the patient.

26. A solution comprising an inhibitor of TNF-α and heparinized blood, which solution is free of active TGF-β or does not contain active TGF-β in an amount greater than endogenous blood levels.

27. The solution of claim 26, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

28. The solution of claim 26, wherein said solution further comprises an analog of adenosine 3′,5′-cyclic monophosphate or an analog of guanosine 3′,5′-cyclic monophosphate.

29. The solution of claim 26, wherein said solution further comprises dibutyryl adenosine 3′,5′-cyclic monophosphate (db cAMP).

30. The solution of claim 26, wherein said solution further comprises 8-bromo-adenosine 3′,5′-cyclic monophosphate (8-bromo-cAMP).

31. A solution consisting of an inhibitor of TNF-α and heparinized blood, which solution is free of active TGF-β or does not contain active TGF-β in an amount greater than endogenous blood levels.

32. The solution of claim 31, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

33. An isolated ex vivo blood vessel or functional portion thereof in contact with a solution comprising an inhibitor of TNF-α, which solution is free of active TGF-β or does not contain active TGF-β in an amount greater than endogenous blood levels.

34. The blood vessel or portion thereof of claim 33, wherein the contacting is at a temperature in the range of about 0.5° C. to about 10° C.

35. The blood vessel or portion thereof of claim 33, wherein the blood vessel is a saphenous vein, a mammary artery, or a radial artery.

36. The blood vessel or portion thereof of claim 33, wherein the blood vessel is a saphenous vein.

37. The blood vessel or portion thereof of claim 33, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

38. The blood vessel or portion thereof of claim 37, wherein the concentration of the inhibitor of TNF-α ranges from about 0.1 μM to 100 μM.

39. The blood vessel or portion thereof of claim 33, wherein said solution comprises heparinized blood, and said solution does not contain active TGF-β or does not contain active TGF-β in an amount greater than endogenous blood levels.

40. The blood vessel or portion thereof of claim 33, wherein said solution is buffered saline further comprising said inhibitor.

41. The blood vessel or portion thereof of claim 33, wherein said solution is the Columbia University solution further comprising said inhibitor.

42. The blood vessel or portion thereof of claim 33, wherein said solution is the Euro-Collins solution further comprising said inhibitor.

43. The blood vessel or portion thereof of claim 33, wherein said solution is the University of Wisconsin solution further comprising said inhibitor.

44. The blood vessel or portion thereof of claim 33, wherein said solution is the low-potassium dextran glucose solution further comprising said inhibitor.

45. The blood vessel or portion thereof of claim 33, wherein said solution is the CELSIOR™ solution further comprising said inhibitor.

46. The blood vessel or portion thereof of claim 33, wherein said solution further comprises an analog of adenosine 3′,5′-cyclic monophosphate or an analog of guanosine 3′,5′-cyclic monophosphate.

47. The blood vessel or portion thereof of claim 46, wherein said solution further comprises dibutyryl adenosine 3′,5′-cyclic monophosphate (db cAMP).

48. The blood vessel or portion thereof of claim 46, wherein said solution further comprises 8-bromo-adenosine 3′,5′-cyclic monophosphate (8-bromo-cAMP).

49. The blood vessel or portion thereof of claim 33, wherein said solution further comprises:
(a) a vasodilator in an amount sufficient to maintain vascular homeostasis, wherein the vasodilator is selected from the group consisting of: adenosine 3′,5′-cyclic monophosphate analogues, guanosine 3′,5′-cyclic monophosphate analogues, nitroglycerin, and pertussis toxin;
(b) a sugar in an amount sufficient to support intracellular function and maintenance of cellular bioenergetics;
(c) magnesium ions in an amount sufficient to support intracellular function and maintenance of cellular bioenergetics;
(d) a macromolecule of molecular weight greater than 20,000 daltons in an amount sufficient to maintain endothelial integrity and cellular viability;
(e) potassium ions in a concentration greater than about 110 mM; and
(f) a buffer in an amount sufficient to maintain the average pH of the blood vessel or portion thereof at about the physiologic pH value,
wherein said solution is free of active TGF-0 or does not contain active TGF-β in an amount greater than endogenous blood levels.

50. The blood vessel or portion thereof of claim 33, which is a human blood vessel or portion thereof.

51. A container containing the blood vessel or portion thereof of claim 33.

52. A method of preserving a blood vessel comprising contacting an isolated blood vessel or portion thereof ex vivo with a solution comprising an inhibitor of TNF-α, which solution is free of active TGF-β or does not contain active TGF-β in an amount greater than endogenous blood levels.

53. The method of claim 52, wherein said solution comprises heparinized blood.

54. The method of claim 52, wherein said contacting is for a time period not longer than four hours.

55. The method of claim 52, wherein the temperature of the solution ranges from about 0.5° C. to about 10° C.

56. The method of claim 52, which further comprises a step of removing the solution from contact with the blood vessel or portion thereof.

57. The method of claim 56, wherein said removing step comprises flushing the blood vessel or portion thereof with a second solution lacking said inhibitor.

58. The method of claim 52, wherein the blood vessel is a saphenous vein, a mammary artery, or a radial artery; and the vascular graft is a coronary artery bypass graft.

59. The method of claim 58, wherein the blood vessel is a saphenous vein.

60. The method of claim 52, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

61. The method of claim 60, wherein the concentration of the inhibitor of TNF-α ranges from about 0.1 μM to 100 μM.

62. The method of claim 52, wherein said solution further comprises buffered saline.

63. The method of claim 52, wherein said solution is the Columbia University solution further comprising said inhibitor.

64. The method of claim 52, wherein said solution is the Euro-Collins solution further comprising said inhibitor.

65. The method of claim 52, wherein said solution is the University of Wisconsin solution further comprising said inhibitor.

66. The method of claim 52, wherein said solution is the low-potassium dextran glucose solution further comprising said inhibitor.

67. The method of claim 52, wherein said solution is the CELSIOR™ solution further comprising said inhibitor.

68. The method of claim 52, wherein said solution further comprises an analog of adenosine 3′,5′-cyclic monophosphate or an analog of guanosine 3′,5′-cyclic monophosphate.

69. The method of claim 68, wherein said solution further comprises dibutyryl adenosine 3′,5′-cyclic monophosphate (db cAMP).

70. The method of claim 68, wherein said solution further comprises 8-bromo-adenosine 3′,5′-cyclic monophosphate (8-bromo-cAMP).

71. The method of claim 52, wherein said solution further comprises:
(a) a vasodilator in an amount sufficient to maintain vascular homeostasis, wherein the vasodilator is selected from the group consisting of: adenosine 3′,5′-cyclic monophosphate analogues, guanosine 3′,5′-cyclic monophosphate analogues, nitroglycerin, and pertussis toxin;
(b) a sugar in an amount sufficient to support intracellular function and maintenance of cellular bioenergetics;
(c) magnesium ions in an amount sufficient to support intracellular function and maintenance of cellular bioenergetics;
(d) a macromolecule of molecular weight greater than 20,000 daltons in an amount sufficient to maintain endothelial integrity and cellular viability;
(e) potassium ions in a concentration greater than about 110 mM; and
(f) a buffer in an amount sufficient to maintain the average pH of the blood vessel or portion thereof during said contacting at about the physiologic pH value.

72. The method of claim 52 wherein the blood vessel or portion thereof is a human blood vessel or portion thereof.

73. A method of preserving a blood vessel comprising contacting an isolated blood vessel or portion thereof ex vivo with a solution consisting of an inhibitor of TNF-α, which solution is free of active TGF-β or does not contain active TGF-β in an amount greater than endogenous blood levels, said solution selected from the group consisting of heparinized blood, buffered saline, the Columbia University solution, the Euro-Collins solution, the University of Wisconsin solution, the low-potassium dextran glucose solution and the CELSIOR™ solution.

74. The method of claim 73, wherein the temperature of the solution ranges from about 0.5° C. to about 10° C.

75. The method of claim 73, wherein said contacting is for a time period not longer than four hours.

76. The method of claim 73, which further comprises a step of removing the solution from contact with the blood vessel or portion thereof.

77. The method of claim 76, wherein said removing step comprises flushing the blood vessel or portion thereof with a second solution lacking said inhibitor.

78. The method of claim 73, wherein the blood vessel is a saphenous vein, a mammary artery, or a radial artery; and the vascular graft is a coronary artery bypass graft.

79. The method of claim 78, wherein the blood vessel is a saphenous vein.

80. The method of claim 73, wherein said inhibitor is selected from the group consisting of etanercept (ENBREL™), infliximab (REMICADE™), adalimumab (HUMIRA™), thalidomide, PEG-sTNF-RI, DN-TNF, and a mixture of any two or more of the foregoing.

81. The method of claim 80, wherein the concentration of the inhibitor of TNF-α ranges from about 0.1 μM to 100 μM.

82. The method of claim 73, wherein the blood vessel or portion thereof is a human blood vessel or portion thereof.

83. The method of claim 1, wherein the patient is a human.

84. The method of claim 1, wherein the contacting comprises immersing, infusing, flushing, or perfusing.

85. The method of claim 1, wherein the blood vessel is one or a combination of:
the internal mammary artery, the radial artery, the internal thoracic artery, right gastroepiploic artery, inferior epigastric artery, or the saphenous vein.

86. The method of claim 85, wherein the blood vessel is the saphenous vein.

87. A method for performing a coronary artery bypass graft in a patient comprising,
(a) removing from contact with a blood vessel or functional portion thereof a solution comprising an inhibitor of TNF-α, which solution is free of active TGF-β or does not contain active TGF-β in an amount greater than endogenous blood levels; and
(b) grafting the blood vessel or functional portion thereof into the patient so as to serve as a coronary bypass graft.

88. The method of claim 87, wherein the patient is a human patient.
